(19) United States
(12) Patent Application Publication
(10) Pub. No.: US 2013/0236892 A1
(43) Pub. Date: Sep. 12, 2013

(54) MEANS AND METHODS FOR THE DETECTION OF A PREDISPOSITION OF A FEMALE SUBJECT TO RECURRENT PREGNANCY LOSS (RPL), PREECLAMPSIA (PE) AND/OR FETAL GROWTH RESTRICTION (FGR)

(71) Applicants: Arseni Markov, Muenster (DE); Nadja Bogdanova, Muenster (DE); Volker Gerke, Altenberge (DE)

(72) Inventors: Arseni Markov, Muenster (DE); Nadja Bogdanova, Muenster (DE); Volker Gerke, Altenberge (DE)

(21) Appl. No.: 13/819,291

(22) PCT Filed: Aug. 26, 2011
(86) PCT No.: PCT/EP2011/064721

§ 371 Date: May 24, 2013

Related U.S. Application Data
(60) Provisional application No. 61/381,561, filed on Sep. 10, 2010.

(30) Foreign Application Priority Data
Aug. 27, 2010 (EP) 10008954.9
Aug. 29, 2010 (EP) 10008970.5
Sep. 10, 2010 (EP) 10176114.6

(51) Int. Cl. C12Q 1/68

(52) U.S. Cl. CPC C12Q 1/6883
USPC 435/6.11; 536/24.1

(57) ABSTRACT

The present invention relates to a method for diagnosing or detecting a predisposition of a female subject to recurrent pregnancy loss (RPL), preeclampsia (PE) and/or fetal growth restriction (FGR), comprising examining the human annexin A5 (ANXA5) promoter in a sample obtained from the intended biological father or the biological father and to detect nucleotide exchanges therein, wherein the presence of the nucleotide exchanges defined in (i) and/or (ii) indicates a predisposition of said female subject to recurrent pregnancy loss (RPL), preeclampsia (PE) and/or fetal growth restriction (FGR). The present invention also relates to a nucleic acid sequence comprising a human annexin A5 (ANXA5) promoter, which promoter comprises specific nucleotide exchanges defined herein, for use in the methods disclosed herein. The present invention further relates to a kit for use in the diagnostic methods disclosed herein.

The present invention relates to a method for diagnosing or detecting a predisposition of a female subject to recurrent pregnancy loss (RPL), preeclampsia (PE) and/or fetal growth restriction (FGR), comprising examining the human annexin A5 (ANXA5) promoter in a sample obtained from the intended biological father or the biological father and to detect nucleotide exchanges therein, wherein the presence of the nucleotide exchanges defined in (i) and/or (ii) indicates a predisposition of said female subject to recurrent pregnancy loss (RPL), preeclampsia (PE) and/or fetal growth restriction (FGR). The present invention also relates to a nucleic acid sequence comprising a human annexin A5 (ANXA5) promoter, which promoter comprises specific nucleotide exchanges defined herein, for use in the methods disclosed herein. The present invention further relates to a kit for use in the diagnostic methods disclosed herein.

Annexin A5 shares the essential annexin family properties, but is a member that can be found extracellularly [1]. It can inhibit coagulation by various mechanisms [2, 3]. The regulation of ANXA5 gene expression in specific tissues is largely unknown. Annexin A5 is distributed abundantly and ubiquitously, mostly in kidney, liver and placenta [4]. The human ANXA5 gene generates several transcripts and has a complex promoter [5]. Annexin A5 is also known as placental anticoagulant protein. Lowered annexin A5 expression on placental trophoblast villi has been detected in the presence of antiphospholipid antibodies [6] and has also been confirmed immunohistochemically in patients with preeclampsia (PE) [7]. Recently, we observed that a sequence variation (M2 haplotype), reducing the activity of the ANXA5 gene promoter, represents a risk factor for recurrent pregnancy loss (RPL) [8]. The expression of ANXA5 mRNA in placentas from M2 haplotype carriers was decreased in comparison to normal controls, and in women with obstetric complications (PE and fetal growth restriction, FGR) it was lower than in a control group without pregnancy complications [9]. A more recent study on the role of M2 in RPL and other obstetric complications corroborated our initial findings and revealed an elevated pregnancy-related hypertensive disorders risk, together with a higher bearing on early fetal loss events [10].

The technical problem underlying the present invention is to provide means and methods for diagnosing a predisposition of females to recurrent pregnancy loss (RPL), preeclampsia (PE) and/or fetal growth restriction (FGR).

The present invention addresses this need and provides methods for diagnosing the above mentioned diseases in females.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein. Unless otherwise indicated, the term “at least” preceding a series of elements is to be understood to refer to every element in the series. At least one includes for example, one, two, three, four, or five or even more.

Those skilled in the art will recognize, or be able to ascertain using no more than routine experimentation, many equivalents to the specific embodiments of the invention described herein. Such equivalents are intended to be encompassed by the present invention. Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

In the present work we aimed to trace the allele specific expression of ANXA5 mRNA in placentas carrying the M2 haplotype, conferring higher recurrent pregnancy loss risk, in order to verify directly the role of M2 in the relevant organ. The M2 allele in heterozygous placentas results in an average of 42% reduced ANXA5 mRNA levels as compared to the normal allele. Protein levels in these samples show considerable variations, impossible for statistical interpretation. The M2 allele of ANXA5 can be linked to reduced mRNA levels in heterozygous placentas and could result in more confined protein levels (lowered expression dynamics) of annexin A5.

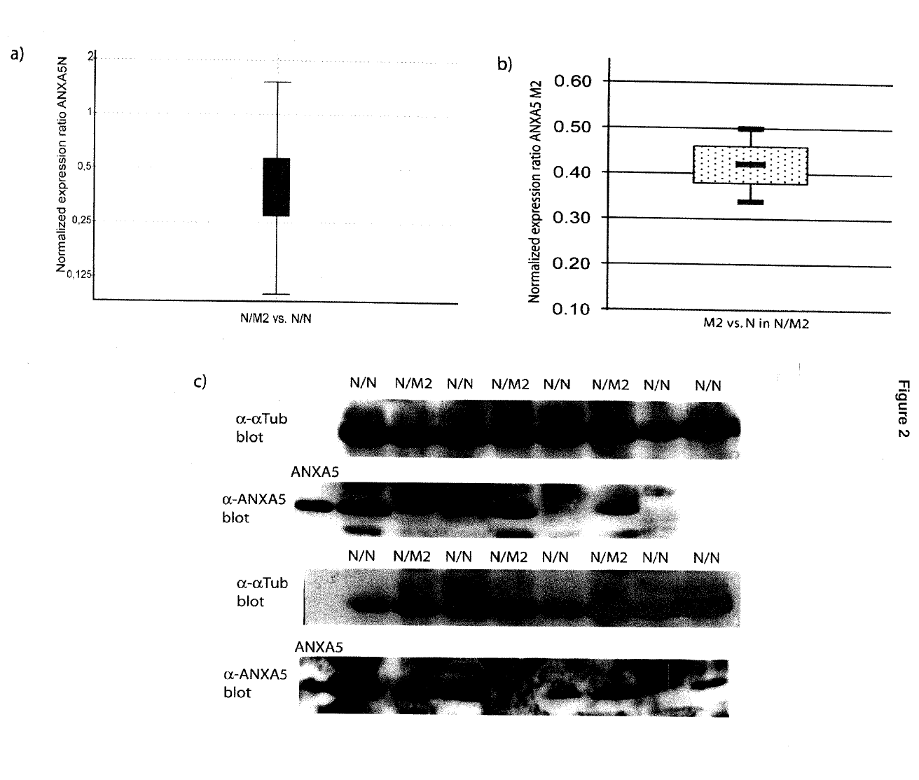

The ANXA5 protein levels in N/N and N/M2 placenta samples were determined as follows. Normalized protein levels in 30 μg of extracted protein were evaluated in N/M2 samples and compared to the N/N samples, with loading control (alpha-tubulin) and annexin A5 detection (FIG. 2c). Protein quantification in both sample groups (N/N vs. N/M2 placentas) was impossible to interpret statistically because of large intragroup variations, at, or several orders above detection limit. In a recent work Sifakis et al. [16] demonstrate significant differences in mRNA expression between normal and FGR pregnancies, and no such differences in protein levels, but the authors did not genotype their samples for M2/ANXA5. Since endogenous expression is not the only source of ANXA5 in placental samples, heterogeneity of tissue sections is crucial to consider, when estimating protein levels. The importance of timing and topical expression in placental microenvironments remains largely unknown. The recorded two-fold reduced range of protein concentrations in FGR samples [16] might reflect disturbed expression, due to the lower responsiveness of the M2 promoter allele. Broader expression dynamics might be potentially needed in enhanced vs. more enhanced anti-coagulation microenvironments of placenta tissue during fetal development.

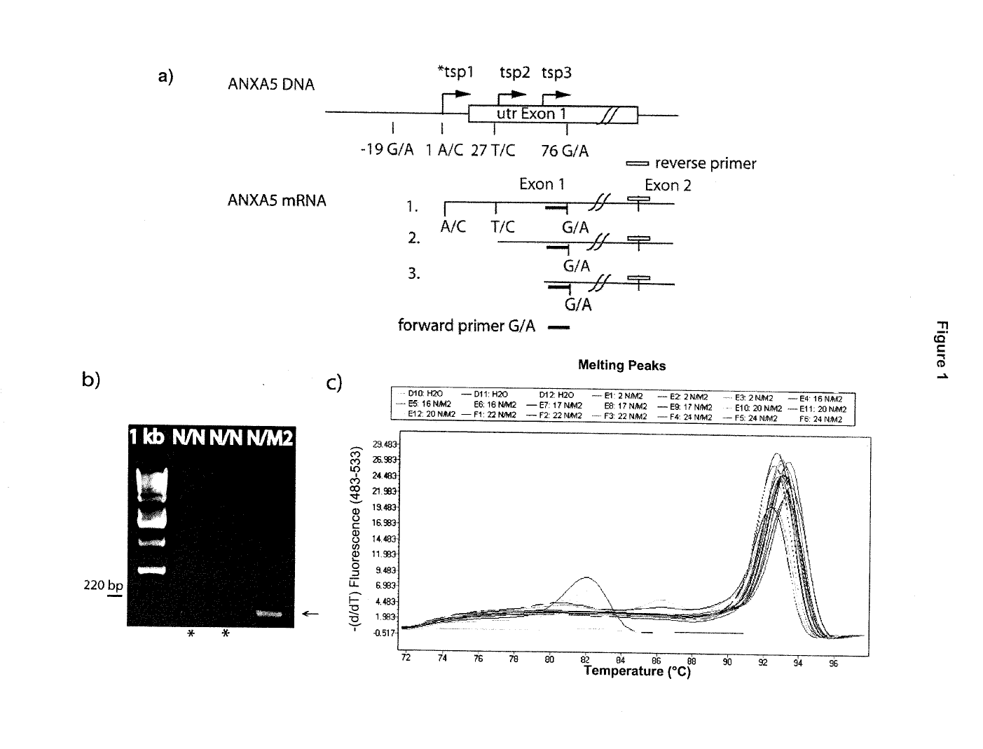

The present study complied with the ethical guidelines of all the institutions involved. Informed consent was obtained from all subjects examined. It is likewise envisaged that informed consent is obtained from all subjects (or their legal guardian) which are to be examined in accordance with the methods of the present invention. The ANXA5 allelic mRNA quantification in placenta samples was conducted as follows. Specific ANXA5 mRNA amplification formats for the normal (N) and haplotype M2 (M2) alleles utilized primers with G/A variation of the last nucleotide, corresponding to the last substitution of the M2 haplotype, 76G>A [8], shared by all detected transcripts of ANXA5 (FIG. 1a). An oligonucleotide bridging exons 1 and 2 served as a reverse primer. Allele specific amplification of this format was verified at respective annealing temperatures by direct product visualization (FIG. 1b) and melting curve analysis (FIG. 1c). The average expression of N allele in N/M2 heterozygous placentas (FIG. 2a) is about 0.4 of the expression in normal homozygote placentas, with a max. value of 0.52 in the middle 50% of observations (p=0.000, randomization test). This is indicative of no allelic compensation of the reduced M2 ANXA5 mRNA through N ANXA5 mRNA. Next, reduced expression of the M2 allele in heterozygous placentas was verified through comparison of the M2 to N expression (FIG. 2b) and it was 0.42 normal allele levels, with a max. value of 0.46 in the interquartile range. This corroborates previous results on the measured M2 promoter activity, which was 37-42% of the normal allele [8]. One sample, showing atypically low M2 expression of 0.01.N with N expression in the normal range was excluded from the statistical evaluation.

Much to our surprise, M2 ANXA5 mRNA is reduced in all N/M2 samples, regardless of the source of the M2 allele (in one discordant genotype sample M2 is of maternal, in the other of paternal origin). The finding that the M2 ANXA5 mRNA is also reduced when the biological father of the respective embryo was the donor of the ANXA5 risk haplotype M2 was unexpected. In particular, it could not have been predicted that the M2 haplotype as such is already predictive for recurrent pregnancy loss (RPL), preeclampsia (PE) and/or fetal growth restriction (FGR). The present inventors, however, have demonstrated that this is obviously the case, i.e. that already the risk haplotype M2 as such is predictive for recurrent pregnancy loss (RPL), preeclampsia (PE) and/or fetal growth restriction (FGR) in a female subject. It is thus possible to test, either alternatively to the biological mother or additionally to the biological mother, the biological father or the embryo as such. The source of the genetic material which has to be analyzed in order to test the predisposition of the female subject is consequently no longer limited to the biological mother (i.e. the biological mother of the embryo) but, in view of the contribution of the present invention, now also extends to the biological father of the embryo, or even the embryo itself.

The above findings in regard to the risk haplotype M2 should also apply to the risk haplotype M1, as it was already shown that the risk haplotype M1 is also predictive for recurrent pregnancy loss (RPL), but to a lesser extent—see in particular WO2006/053725 and [8].

As already disclosed in WO2006/053725 and [8], the risk haplotype M2 which can be detected in the human ANXA5 promoter, is characterized by the following four nucleotide exchanges: (1) G to A at a position which corresponds to nucleotide 186 of SEQ ID No. 2, (2) A to C at a position which corresponds to nucleotide 203 of SEQ ID No. 2, (3) T to C at a position which corresponds to nucleotide 229 of SEQ ID No. 2, and (4) G to A at a position which corresponds to nucleotide 276 of SEQ ID No. 2.

The risk haplotype M1 which can be detected in the human ANXA5 promoter, is also disclosed in WO2006/053725 and [8] and is characterized by the following two nucleotide exchanges (2) A to C at a position which corresponds to nucleotide 203 of SEQ ID No. 2, and (3) T to C at a position which corresponds to nucleotide 229 of SEQ ID No. 2.

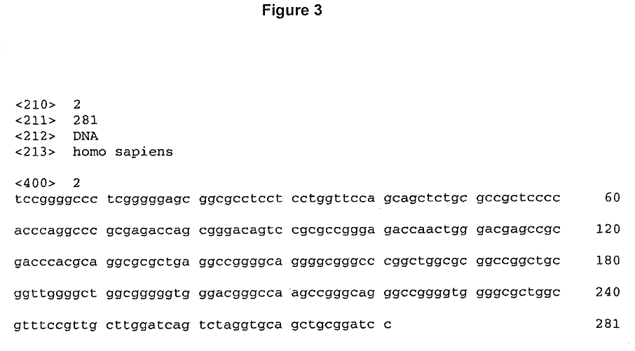

SEQ ID No. 2 as used herein and depicted in FIG. 3 corresponds to SEQ ID No. 2 disclosed in WO2006/053725. Said SEQ ID No 2 depicts an ANXA5 promoter structure as disclosed in Carcedo (2001), Biochem. J. 356, 571-579).

Means and methods to determine and/or to detect these risk haplotypes are well-known (see for example WO2006/053725 and [8]) and additionally disclosed in detail herein.

The present invention relates in one embodiment to a method for diagnosing or detecting a predisposition of a female subject to recurrent pregnancy loss (RPL), preeclampsia (PE) and/or fetal growth restriction (FGR), comprising:

(a) examining the human annexin A5 (ANXA5) promoter in a sample obtained from the intended biological father or the biological father to detect the following nucleotide exchanges
(i)
(1) G to A at a position which corresponds to nucleotide 186 of SEQ ID No. 2,
(2) A to C at a position which corresponds to nucleotide 203 of SEQ ID No. 2
(3) T to C at a position which corresponds to nucleotide 229 of SEQ ID No. 2, and
(4) G to A at a position which corresponds to nucleotide 276 of SEQ ID No. 2;
or
(ii)
(2) A to C at a position which corresponds to nucleotide 203 of SEQ ID No. 2, and
(3) T to C at a position which corresponds to nucleotide 229 of SEQ ID No. 2,
(b) determining whether the nucleotide exchanges defined in (i) and/or (ii) are present,
wherein the presence of the nucleotide exchanges defined in (i) and/or (ii) indicates a predisposition of said female subject to recurrent pregnancy loss (RPL); and wherein the presence of the nucleotide exchanges defined in (i) indicates a predisposition of said female subject to preeclampsia (PE) and/or fetal growth restriction (FGR).

It will be understood that in the context of the embodiments described herein, the female subject was made pregnant by the herein mentioned “biological father”. Thus, as used herein, the term “biological father” means the biological father of the human embryo of the herein defined female subject. In some embodiments, the female subject is already pregnant and is therefore the biological mother—in other embodiments the female subject is not yet pregnant and is therefore the intended biological mother.

The term “intended biological father” therefore means that the female subject is not yet made pregnant by the human male subject, but that it is intended that the human female subject will be made pregnant by said human male subject. During that time, also the female subject is the “intended” biological mother. Once the female subject was made pregnant by said human male subject, the “intended biological father” becomes the “biological father” and the “intended biological mother” becomes the “biological mother”.

As mentioned herein before, the present invention is based on the surprising finding that the M2 ANXA5 mRNA is reduced in all N/M2 samples, regardless of the source of the M2 allele, i.e. it is was shown that it is also possible to test the predisposition of a pregnant or non-pregnant female subject to recurrent pregnancy loss (RPL), preeclampsia (PE) and/or fetal growth restriction (FGR), by way of testing a sample obtained from the biological father or the intended biological father.

The methods of the present invention therefore encompass situations wherein the female subject is not yet made pregnant by the intended biological father, i.e. the female and/or the intended biological mother and the intended biological father plan to test the predisposition of the female subject to recurrent pregnancy loss (RPL), preeclampsia (PE) and/or fetal growth restriction (FGR), prior to the pregnancy. This includes for example couples which plan to have a baby or females which plan to become pregnant, either by natural procreation or by in vitro fertilization.

The methods of the present invention further encompass situations wherein the female subject is already pregnant. In such cases, it might still be wanted to test the predisposition of the female subject to recurrent pregnancy loss (RPL), preeclampsia (PE) and/or fetal growth restriction (FGR), either by way of testing a sample of the biological father and/or by way of testing an embryonic sample of the embryo as such (for example by way of chorion biopsy or by way of amniocentesis, both resulting in samples of embryonic origin). Additionally, but not exclusively, it is also envisaged to test the (intended) biological mother.

Provided that the fertilization is conducted in vitro, i.e. by way of an in vitro fertilization, it is also envisaged to analyse a single cell sample obtained before or during the morula stage of the in vitro fertilized embryo, prior to its implantation into said female subject.

The “morula stage” denotes the 16 cell stage of human embryogenesis. “Before or during the morula stage” means that it is also envisaged to obtain one single cell prior to the 16-cell stadium, for example during the 6-8 cell stadium of human embryogenesis.

In vitro fertilisation (IVF) is a well known process by which egg cells are fertilised by sperm outside the womb, in vitro.

It is also envisaged that females which intend to become pregnant by a sperm donor, make use of the methods of the preset invention in order to test their predisposition to recurrent pregnancy loss (RPL), preeclampsia (PE) and/or fetal growth restriction (FGR) by way of testing the respective sperm donor sample before it is used for the in vitro fertilization of the respective female subject. The present invention and in particular the methods of the present invention therefore also encompass a stratification method for selecting a sperm donor, which is no carrier of the risk haplotype M1 or M2. Thus, by way of the methods of the present invention it is possible to detect and subsequently select a sperm donor who, in all likelihood, will not contribute to the predisposition of the respective female subject to recurrent pregnancy loss (RPL), preeclampsia (PE) and/or fetal growth restriction (FGR). These stratification methods make particularly sense when a maternal sample of the mother was already tested to be no carrier of the risk haplotype M1 or M2, because in such cases it is of importance to test whether the intended biological father (for example the sperm donor) is carrier of the mentioned risk haplotypes. If so, then it might be reasonable to select a different sperm donor, preferably sperm donor who is also no carrier of the risk haplotypes M1 or M2.

In the same way, it is reasonable and therefore particularly envisaged in the embodiments of the present invention to test the intended biological father or the biological father in situation where the biological mother (or the intended biological mother) is no carrier of the risk haplotype M1 or M2 (as tested in a maternal sample). As demonstrated for the first time by the present invention, the M2 risk haplotype mRNA is reduced in all N/M2 samples regardless of the source of the risk haplotype allele, i.e. both the mother but also the father can contribute to a predisposition of the female subject to recurrent pregnancy loss (RPL), preeclampsia (PE) and/or fetal growth restriction (FGR). Accordingly, even if the female subject as such is no carrier of the risk haplotype (tested in a maternal sample), the biological father and/or the intended biological father can still contribute to the above mentioned predisposition and should, therefore, be tested as well.

Provided that either the biological father of the embryo or the biological mother of the embryo is a heterozygous carrier of the risk haplotype, it is also possible to test a sample of said embryo (e.g. a chorion biopsy sample or a single cell sample described herein) in order to test whether it is carrier of the risk haplotype or not (for example in case of an in vitro fertilization). Provided that either the biological mother or the biological father is a homozygous carrier of the risk haplotype, it appears unnecessary to test the embryo as well, as the heterozygous presence of the risk haplotypes M1 or M2 is already indicative for a predisposition of the respective female subject to recurrent pregnancy loss (RPL), preeclampsia (PE) and/or fetal growth restriction (FGR). Provided that the biological father) is untraceable or unknown, and further that the female subject (in that case the biological mother) is no carrier of the M1 or M2 risk haplotype, one might still want to test the predisposition of the respective female subject to recurrent pregnancy loss (RPL), preeclampsia (PE) and/or fetal growth restriction (FGR) —in that case it is envisaged to test a sample which originates from the embryo (e.g. chorion biopsy and/or amniocentesis sample—it will be understood, however, that an embryonic sample should preferably not be obtained solely because the female subject intends to test its predisposition to recurrent pregnancy loss (RPL), preeclampsia (PE) and/or fetal growth restriction (FGR) —it is rather envisaged to test in samples which originate from the embryo only then, when such samples are already at hand for other reasons).

The female subject, biological father and/or the intended biological father described herein are preferably Eurasian.

Recurrent pregnancy loss (RPL) is typically characterized as the occurrence of two or more pregnancies that end in miscarriage of the fetus. Said two or more pregnancies occur either consecutively or intermittently, consecutively being preferred.

Pre-eclampsia (PE) is a medical condition in which hypertension arises in pregnancy (pregnancy-induced hypertension).

Fetal growth restriction (or Fetal growth retardation) is a condition in which a fetus does not grow appropriately. FGR should be suspected for example when the fundal height is more than 3 cm less than predicted.

It is envisaged that in the methods of the present invention, said sample obtained from the intended biological father and/or the biological father is blood sample, a sperm sample, a tissue sample, or a cell sample. It will be understood that any biological sample will be suitable as long as the respective sample contains genetic material which allows the detection/diagnosis which are subject of the methods of the present invention. Such a sample may be obtained via biopsy such as needle biopsy, surgical biopsy, via any kind of smear technique, for example by use of a buccal swab, etc. or others. The skilled person is well aware of further means and methods enabling him or her to obtain a sample containing genetic material from a human subject.

It will be understood that the biological sample of the present invention is of non-maternal origin. “Non-maternal” means that the sample comprises no or essentially no contamination with genetic material obtained from the (intended) biological mother (the female subject) of the present invention. “Essentially no” thereby means that the contamination with genetic material obtained from the (intended) biological mother (the female subject) of the present invention can be tolerated if it does not prevent the reliable detection of the risk-haplotype in the sample of non-maternal origin. The skilled person is well-aware how to avoid or circumvent such contaminations—the corresponding standards are for example summarized in the “General standards and guidelines for prenatal testing are available from the American College of Medical Genetics (2006 Edition of Standards and guidelines for clinical genetics laboratories, http://www.acmg.net/Pages/ACMG_Activities/stds-2002/g.htm”.

A maternal sample however does not exclude that the sample is obtained from the female subject of the present invention—a chorion biopsy or an amniocentesis sample is for example obtained from the female subject but it is a non-maternal sample provided that it does not contain relevant amounts of cells (genetic material) from the respective female subject). “Genetic material” includes all kinds of nucleic acid sequences which can be obtained from the biological samples of the present invention and allow for the detection of the risk-haplotype M1 or M2 by standard nucleic acid detection techniques. A non-maternal sample is in particular a sample which is of fetal or paternal origin—it will be understood that the respective “father” who is the donor of the paternal sample is the (intended) biological father described herein, whereas the “embryo” (donor of the fetal sample) is the embryo of the female subject.

“Maternal sample” as used herein thus means a sample which exclusively reflects the genotype of the female subject. For example, blood obtained from the female subject is a maternal sample, while a sample obtained via chorion biopsy or amniocentesis or the like is no maternal sample (or a sample of non-maternal origin).

As documented in WO 2006/053725, a genetic variant in the promoter region of ANXA5 (BamHI) was surprisingly found in the annexin A5 (ANXA5) gene among women who were examined for different hereditary thrombosis genetic defects because of repeated pregnancy loss. This variant consists of four nucleotide substitutions, which are inherited as a haplotype. These four changes are important for the activity of the annexin A5 promoter and result in a reduced gene expression. As defined herein, of particular relevance in this respect are four point mutations characterized as “−19 G to A”, “1 A to C”, “27 T to C” and “76 G to A”, whereby “G” denotes guanine, “C” denotes cytosine, “A” denotes adenine and “T” thymine.

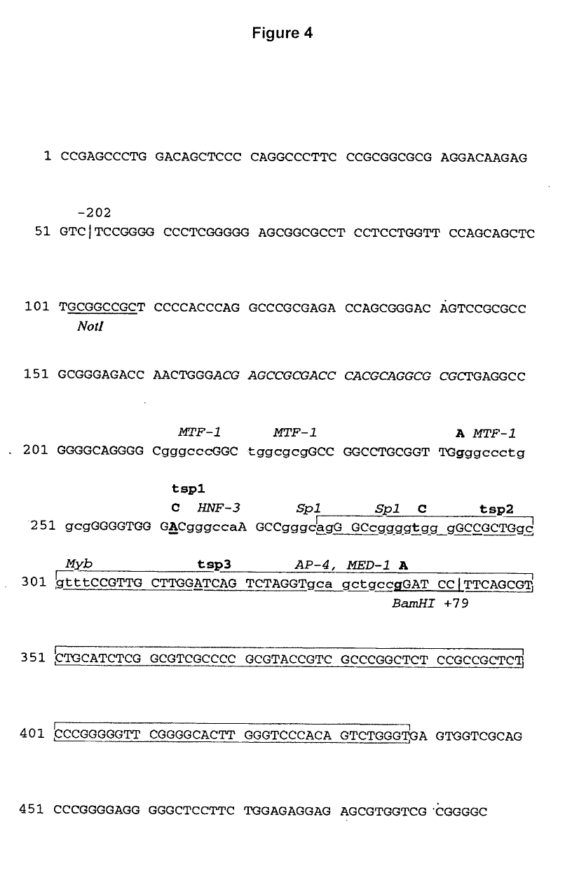

The positions “−19”, “1”, “27” and “76” of the mutations/substitutions described herein relate to the numbering of the sequence as given in appended FIG. 4, depicting the ANXA5 core promoter structure. The numbering in particular relates to the first transcription start point of the gene (tsp 1, as indicated “+1”). However, the corresponding substitutions/mutations are also defined in relation to specific sequences representing the ANXA5 promoter, and given in particular in SEQ ID NO: 2 (an ANXA5 promoter structure as disclosed in Carcedo (2001), Biochem. J. 356, 571-579). Said SEQ ID No 2 is depicted herein in FIG. 3.

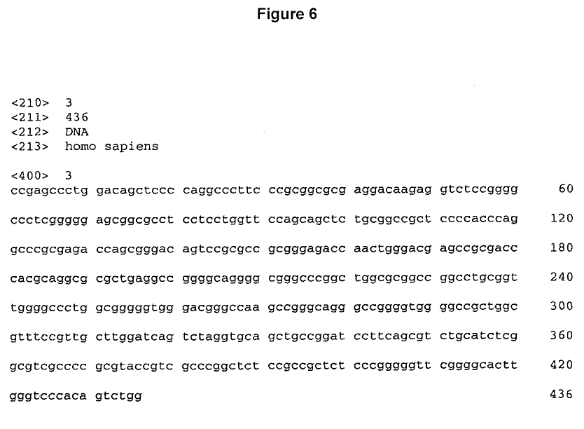

SEQ ID No. 2 as depicted herein corresponds in addition to SEQ ID No. 2 as depicted in WO2006/053725, which is included herein in its entirety. Further sequences which may be used alternatively for the determination of the nucleotide positions in the human ANXA5 promoter have already been described in the cited WO2006/053725, and in particular on pages 3 and 4 of said document (which are incorporated herein by way of reference thereto). It cannot be excluded that the risk conferred by haplotypes M2 or M1 could be modified to some extent by other SNPs occurring in the promoter region of ANXA5 gene, as defined in FIGS. 4 and 6 (SEQ ID No. 3), in other, not Eurasian populations. These alternative and/or additional SNP(s) could be identified in representative samples of these populations through nucleic acids sequencing of the ANXA5 promoter region, a technique well known to the skilled person and also described herein.

In a further aspect, the present invention relates to a method for diagnosing or detecting a predisposition of a female subject to recurrent pregnancy loss (RPL), preeclampsia (PE) and/or fetal growth restriction (FGR), comprising:

(a) examining the human annexin A5 (ANXA5) promoter in a chorion biopsy or amniocentesis sample obtained from said female to detect the following nucleotide exchanges
(i)
(1) G to A at a position which corresponds to nucleotide 186 of SEQ ID No. 2,
(2) A to C at a position which corresponds to nucleotide 203 of SEQ ID No. 2
(3) T to C at a position which corresponds to nucleotide 229 of SEQ ID No. 2, and
(4) G to A at a position which corresponds to nucleotide 276 of SEQ ID No. 2;
or
(ii)
(2) A to C at a position which corresponds to nucleotide 203 of SEQ ID No. 2, and
(3) T to C at a position which corresponds to nucleotide 229 of SEQ ID No. 2,
(b) determining whether the nucleotide exchanges defined in (i) and/or (ii) are present,
wherein the presence of the nucleotide exchanges defined in (i) and/or (ii) indicates a predisposition of said female subject to recurrent pregnancy loss (RPL); and wherein the presence of the nucleotide exchanges defined in (i) indicates a predisposition of said female subject to preeclampsia (PE) and/or fetal growth restriction (FGR).

The above mentioned chorion biopsy and/or amniocentesis should preferably not be triggered by the wish of the female subject and/or the biological father (and/or the respective couple) to test solely for the predisposition of the female subject to recurrent pregnancy loss (RPL), preeclampsia (PE) and/or fetal growth restriction (FGR).

The present invention further relates to a method for diagnosing or detecting a predisposition of a female subject to recurrent pregnancy loss (RPL), preeclampsia (PE) and/or fetal growth restriction (FGR), comprising:

(a) examining the human annexin A5 (ANXA5) promoter in a single cell sample obtained before or during the morula stage of an in vitro fertilized embryo prior to its implantation into said female subject to detect the following nucleotide exchanges
(i)
(1) G to A at a position which corresponds to nucleotide 186 of SEQ ID No. 2,
(2) A to C at a position which corresponds to nucleotide 203 of SEQ ID No. 2
(3) T to C at a position which corresponds to nucleotide 229 of SEQ ID No. 2, and
(4) G to A at a position which corresponds to nucleotide 276 of SEQ ID No. 2;
or
(ii)
(2) A to C at a position which corresponds to nucleotide 203 of SEQ ID No. 2, and
(3) T to C at a position which corresponds to nucleotide 229 of SEQ ID No. 2,
(b) determining whether the nucleotide exchanges defined in (i) and/or (ii) are present,
wherein the presence of the nucleotide exchanges defined in (i) and/or (ii) indicates a predisposition of said female subject to recurrent pregnancy loss (RPL); and wherein the presence of the nucleotide exchanges defined in (i) indicates a predisposition of said female subject to preeclampsia (PE) and/or fetal growth restriction (FGR).

As mentioned before, it is envisaged to test the intended biological father or the biological father in situation where the biological mother (or the intended biological mother) is no carrier of the risk haplotype M1 or M2 (as tested in a maternal sample). In the same way, it is also envisaged to test the intended biological mother or biological mother in situations where the biological father (or the intended biological father) is no carrier of the risk haplotype M1 or M2.

It is thus also envisaged that the methods described herein above may further comprise the steps of:

(c) examining the human annexin A5 (ANXA5) promoter in a maternal sample obtained from said female subject to detect the nucleotide exchanges defined in (i) and/or (ii) of any one of the preceding claims, and
(d) determining whether the nucleotide exchanges defined in (i) and/or (ii) of the preceding claims are present.

The above method is particularly useful in situations where the (intended) biological father has already been tested as being “negative”, i.e. the father is no carrier of the risk haplotype M1 or M2.

In a further embodiment, the present invention relates to a method described herein before, wherein said female subject is a subject which has been diagnosed to have a predisposition to recurrent pregnancy loss (RPL), preeclampsia (PE) and/or fetal growth restriction (FGR), by way of examining the human annexin A5 (ANXA5) promoter in a maternal sample obtained from said female to detect the nucleotide exchanges defined in (i) or (ii) of any one of the preceding claims.

In a further embodiment, the present invention relates to a method described hereinbefore, wherein said female subject is a subject which has been diagnosed not to have a predisposition to recurrent pregnancy loss (RPL), preeclampsia (PE) and/or fetal growth restriction (FGR), by way of examining the human annexin A5 (ANXA5) promoter in a maternal sample obtained from said female to detect the nucleotide exchanges defined in (i) or (ii) of any one of the preceding claims.

These methods particularly relate to situations where the risk haplotype was determined in the mother prior to the determination of the risk haplotype in the (intended) biological father or the embryo (the latter either by way of testing a single cell for in the course of an in vitro fertilization or, alternatively by way of testing a sample of embryonic origin, e.g. a chorion biopsy etc.).

The present invention further relates to a method as described hereinbefore, wherein the detection of the nucleotide exchanges defined in

(i)
(1) G to A at a position which corresponds to nucleotide 186 of SEQ ID No. 2,
(2) A to C at a position which corresponds to nucleotide 203 of SEQ ID No. 2
(3) T to C at a position which corresponds to nucleotide 229 of SEQ ID No. 2, and
(4) G to A at a position which corresponds to nucleotide 276 of SEQ ID No. 2;
or
(ii)
(2) A to C at a position which corresponds to nucleotide 203 of SEQ ID No. 2, and
(3) T to C at a position which corresponds to nucleotide 229 of SEQ ID No. 2, is carried out by nucleic acid detection techniques.

“Nucleic acid detection techniques” are well-known to the skilled person and include inter alia any kind of PCR-based techniques or any other suitable technique which allows the identification of the nucleotide exchanges which characterize the risk haplotype M1 and/or M2. Such methods are described herein (see the examples) and are also published for example in [8] or in WO2006/053725.

Said techniques may be selected from the non-limiting group consisting of hybridization techniques, nucleic acid sequencing, PCR, restriction fragment determination, single nucleotide polymorphism (SNPs)-determination, LCR (ligation chain reaction) or restriction fragment length polymorphism (RFLP)-determination, to name some. Corresponding examples and further details may be obtained from standard technical advise literature (like Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989), or Higgins and Hames (Eds.)). As documented in the examples of WO2006/053725, a further suitable method is the restriction fragment determination or the RFLP method, comprising the determination of a BamHI restriction site. As shown in WO2006/053725, the absence (BamHI′) or the presence (BamHI+) of a BamHI restriction site is determined, and is indicative for the absence or presence of a point mutation as defined herein. Details on this method are given in the appended examples of WO2006/053725. In one embodiment, a relevant DNA-stretch may be amplified from genomic DNA by PCR-technology. Potential primers to be employed comprise, but are not limited to, the primers as provided in SEQ ID NO: 22 (ANX5.P.F) and SEQ ID NO: 23 (ANX5.exl.R) of WO2006/053725. The person skilled in the art is readily in the position to deduce further primer pairs or primers to be employed in order to amplify relevant stretches of the herein defined annexin A5 (ANXA5) promoter or of its fragments. After the amplicon is obtained (see also experimental part) it can be digested (restriction digest) with the restriction enzyme BamHI (which can be obtained from various suppliers, inter alia: Roche Applied Science, Mannheim, Germany; MBI Fermentas, St. Leon-Rot, Germany; New England Biolabs, Frankfurt am Main, Germany. Again, details are given in the experimental part. After this digest, to be carried in accordance with methods well-known in the art (see inter alia Sambrook/Russel, 2001, (log.cit.)), further analysis of the BamHI/BamHI+restriction site can be carried out by known techniques, like gel analysis, e.g. agarose gel analysis.

A further technique which is particularly envisaged in the context of the present invention is the SNP detection technique established by IHG Pharmaco. Said technique is sufficiently explained in WO 2006/038037.

Thus, in a preferred embodiment, the detection of the nucleotide exchanges defined in (i) and/or (ii) is carried out by a method for genotyping a target gene sequence associated with an inherited genetic disorder, comprising:
(a) providing a population of induced heteroduplex generator (IHG) molecules corresponding to said target gene sequence, the IHG molecule being a synthetic DNA sequence including at least one nucleotide position which corresponds to a known polymorphic site in the genomic DNA sequence of the target gene sequence and at least one nucleotide substitution, deletion and/or insertion (“identifier”) relative to the genomic sequence at a nucleotide position spaced by a distance of at least one base from the nucleotide position which corresponds to the known polymorphic site and wherein the nucleotide(s) between the nucleotide position which corresponds to the polymorphic site and the identifier are unchanged from the genomic sequence; wherein the at least one identifier comprises an insertion of one or more bases and the IHG molecule is selected to provide improved separation of the resolved bands by a method comprising comparing the separation obtained using the IHG molecule with the separation obtained using a corresponding IHG in which the identifier is not so spaced;
(b) providing a population of the target gene sequence;
(c) combining the respective populations of (a) and (b) under conditions suitable for heteroduplex formation, to obtain induced heteroduplexes between the target gene sequence and an IHG molecule corresponding to said target gene sequence;
(d) resolving the induced heteroduplexes into bands on a suitable support; and
(e) analysing the resolved induced heteroduplexes to determine the genotype of the target gene sequence.

Said technique is, as mentioned before, disclosed in great detail in WO 2006/038037 which is disclosed herein in its entirety (including the respective definitions of the terms used in the embodiment above). It will be understood that said “target gene sequence” is the human ANXA5 promoter or a fragment thereof.

It will be understood that the methods of the present invention are carried out “in vitro” (which equates with “ex vivo”). It is thus envisaged that the methods disclosed herein do preferably not include the step of obtaining the respective sample. Rather, the sample is already obtained and is provided in suitable container, i.e. the sample is preferably contained in a container, which container (including the sample) is then employed in the methods of the present invention.

The present invention also relates to an IHG which can be employed in the IHG method characterized above and explained in WO 2006/038037, i.e. an IHG which is capable of detecting the risk haplotype M1 or M2 by way of the above identified IHG method. The skilled person knows how to design such an IHG, as the document WO 2006/038037 (and thereby also the present specification) provides sufficient guidance in this regard.

In a further embodiment, said IHG is comprised in a buffer. It is preferred that said buffer allows for the formation of heteroduplexes in accordance with the IHG method explained hereinabove and as well as in WO 2006/038037.

The present invention also relates to a nucleic acid sequence comprising a human annexin A5 (ANXA5) promoter or a fragment thereof, which promoter or fragment thereof comprises the nucleotide exchanges defined in

(i)
(1) G to A at a position which corresponds to nucleotide 186 of SEQ ID No. 2,
(2) A to C at a position which corresponds to nucleotide 203 of SEQ ID No. 2
(3) T to C at a position which corresponds to nucleotide 229 of SEQ ID No. 2, and
(4) G to A at a position which corresponds to nucleotide 276 of SEQ ID No. 2;
or
(ii)
(2) A to C at a position which corresponds to nucleotide 203 of SEQ ID No. 2, and
(3) T to C at a position which corresponds to nucleotide 229 of SEQ ID No. 2, for use in a method of the present invention.

Said nucleic acid sequence comprising a human annexin A5 (ANXA5) promoter is exemplified by SEQ ID No. 2, but the invention is in no way limited thereto. Further suitable nucleotide sequences have been disclosed in for example in WO2006/053725 (therein SEQ Id No 1, 3 or 4—see for example the table on page 4, which is also depicted herein below).

| | SEQ ID | | | |
|---|---|---|---|---|
| Substitution | NO: 2 | SEQ ID NO: 1 | SEQ ID NO: 3 | SEQ ID NO: 4 |
| −19 G→ A | 186 | 1259 | 243 | 190 |
| 1 A → C | 203 | 1276 | 262 | 209 |
| 27 T → C | 229 | 1302 | 288 | 235 |
| 76 G→ A | 276 | 1349 | 337 | 284 |

SEQ ID NO: 1 thereby relates, in accordance with the definition in WO2006/053725 to an ANXA5 promoter structure as deposited under gene accession number U0181; NCBI) and as annotated as—human annexin V gene, 5′-untranslated region, exons 1 and 2). Two further annexin V-promoter regions/5′-untranslated regions are defined herein (in accordance with WO2006/053725) as SEQ ID NOS: 3 and 4. The 5′-untranslated region of an annexin V gene may, in context of this invention, be characterized as comprising two specific motifs “A” and “B” which are documented in FIG. 4.

Also comprised in the definition of a nucleic acid sequence comprising a human annexin A5 (ANXA5) promoter of the present invention are nucleic acid molecules which are at least 60%, more preferably at least 70%, more preferably at least 80%, more preferably at least 90% even more preferably 95% and most preferably at least 97% identical to the promoter sequences as shown in any one of SEQ ID NOS: 1 to 4, SEQ ID No. 2 being preferred. “Percent (%) nucleotide sequence identity” is defined as the percentage of nucleotide residues in a candidate sequence (sequence of interest) that are identical with the nucleotide residues in the ANXA5 nucleotide sequence shown in SEQ ID No:1, 2, 3 or 4; SEQ ID No. 2 being preferred, after aligning the sequences and introducing gaps, if necessary, to achieve the maximum percent sequence identity, and not considering any conservative substitutions as part of the sequence identity. Alignment for purposes of determining percent nucleotide sequence identity can be achieved in various ways that are within the skill in the art, for instance, using publically available computer software such as BLAST, ALIGN, or Megalign (DNASTAR) software. Those skilled in the art can determine appropriate parameters for measuring alignment, including any algorithms needed to achieve maximum alignment over the full length of the sequences being compared. The degree of identity is preferably over the entire length with the nucleotide sequence of SEQ ID No:2.

It will be understood, however, that also the respective nucleotide sequences having a certain degree of identity as disclosed hereinabove, still comprise the nucleotide exchanges defined in

(i)
(1) G to A at a position which corresponds to nucleotide 186 of SEQ ID No. 2,
(2) A to C at a position which corresponds to nucleotide 203 of SEQ ID No. 2
(3) T to C at a position which corresponds to nucleotide 229 of SEQ ID No. 2, and
(4) G to A at a position which corresponds to nucleotide 276 of SEQ ID No. 2;
or
(ii)
(2) A to C at a position which corresponds to nucleotide 203 of SEQ ID No. 2, and
(3) T to C at a position which corresponds to nucleotide 229 of SEQ ID No. 2.

The above defined nucleic acid sequence comprising a human annexin A5 (ANXA5) promoter are preferably capable to drive the transcription of the ANXA5 gene or another gene or nucleic acid sequence (e.g. a reporter gene) which is under the control of the respective nucleic acid sequence comprising a human annexin A5 (ANXA5) promoter.

The “fragments of the human annexin A5 (ANXA5) promoter”, however, are not necessarily capable to drive the transcription of the ANXA5 gene or another gene or nucleic acid sequence (for example a reporter gene). These fragments have a length of at least 27 consecutive nucleotides in case of the risk haplotype M1, or a length of at least 91 nucleotides in case of risk haplotype M2. The maximum length of these fragments is preferably less then 50, more preferably less then 35 nucleotides in regard to the risk haplotype M1, and/or less then 120, more preferably less then 100 nucleotides in length as regards risk haplotype M2.

Nucleic acid molecules which are at least 60%, more preferably at least 70%, more preferably at least 80%, more preferably at least 90% even more preferably 95% and most preferably at least 97% identical to the fragments defined herein above, are also envisaged.

Examples of reporter genes are genes which encode luciferase, (green/red) fluorescent protein and variants thereof, like eGFP (enhanced green fluorescent protein), RFP (red fluorescent proteins, like DsRed or DsRed2), CFP (cyan fluorescent protein), BFP (blue fluorescent protein), YFP (yellow fluorescent protein), β-galactosidase, glucose oxidase, maltose binding protein or chloramphenicol acetyltransferase, to name some.

In another aspect, the present invention relates to a nucleic acid sequence which hybridizes to a nucleic acid sequence comprising a human annexin A5 (ANXA5) promoter or a fragment thereof, which promoter or fragment thereof comprises the nucleotide exchanges defined in

(i)
(1) G to A at a position which corresponds to nucleotide 186 of SEQ ID No. 2,
(2) A to C at a position which corresponds to nucleotide 203 of SEQ ID No. 2
(3) T to C at a position which corresponds to nucleotide 229 of SEQ ID No. 2, and
(4) G to A at a position which corresponds to nucleotide 276 of SEQ ID No. 2;
or
(ii)
(2) A to C at a position which corresponds to nucleotide 203 of SEQ ID No. 2, and
(3) T to C at a position which corresponds to nucleotide 229 of SEQ ID No. 2,
for use in a method of the present invention. It will be understood that the respective hybridizing nucleic acid sequence, comprises the above identified nucleotide exchanges defined in (i) or (ii), as well.

The term “hybridize” as used herein refers to conventional hybridization conditions, preferably to hybridization conditions at which 5×SSPE, 1% SDS, 1×Denhardts solution is used as a solution and/or hybridization temperatures are between 35° C. and 70° C., preferably 65° C. After hybridization, washing is preferably carried out first with 2×SSC, 1% SDS and subsequently with 0.2×SSC at temperatures between 35° C. and 70° C., preferably at 65° C. (regarding the definition of SSPE, SSC and Denhardts solution see Sambrook, loc. cit.). Stringent hybridization conditions as for instance described in Sambrook, supra, are particularly preferred. Particularly preferred stringent hybridization conditions are for instance present if hybridization and washing occur at 65° C. as indicated above. Non-stringent hybridization conditions, for instance with hybridization and washing carried out at 45° C., are less preferred.

The term “comprises” includes in the context of the above identified nucleic acid sequences, that these sequences may contain further nucleotides or stretches of nucleotides which are not necessarily identical to the human ANXA5 promoter. Such stretches may resemble artificial hybridization sites for amplification primers (for example universal primers which can be used for the amplification of different nucleic acid sequences), restriction sites, tags, etc. Said further nucleotide nucleotides or stretches of nucleotides are preferably heterologous to the genetic sequences upstream and downstream of the human ANXA5 promoter. “Stretches” includes up to several kpb of consecutive nucleotides, however preferably not exceeding the typical length of a DNA vector, i.e. preferably not exceeding 5 kbp.

All the above mentioned nucleic acid sequences can be used in the methods of the present invention.

The present invention further relates to the above identified nucleic acid sequences, which are under conditions suitable for the detection of said nucleic acid sequence.

The present invention also relates to the above nucleotide sequences which are under conditions suitable for the detection of the human ANXA5 promoter, for example in a human sample described herein.

Provided that the detection is carried out by way of amplification methods such as PCR, the respective “suitable conditions” are adapted to the respective amplification method. “Suitable conditions” therefore typically denotes the presence of the nucleotide sequences described herein, in a buffer system, which buffer system contains as a minimum requirement all necessary elements which allow the detection of the respective nucleic acid as such, or which allow at least the very first step of that detection (which is typically a step of amplification, either via cloning in a host system or by way of a PCR technique, such as a real-time PCR as disclosed in the appended examples). Suitable buffer systems are well known to the skilled person. The skilled person is also well aware which components have to be included in order to amplify or clone a respective nucleic acid sequence.

It is particularly envisaged that the nucleic acid sequences of the present invention are contained in a PCR amplification buffer (preferably also including primers, nucleotides and/or the PCR-enzyme); or in a buffer which allows the formation of heteroduplexes in accordance with the methods disclosed herein, as well as in WO 2006/038037.

In another aspect, the present invention relates to a kit for use in a diagnostic method of the present invention, said kit comprising:
(a) a package insert and/or an imprint indicating that said kit is to be employed in a method of the present invention; and
(b) means to carry out the methods of the present invention; and
(c) optionally the nucleic acid sequence(s) disclosed herein.

The term “package insert” is used to refer to instructions customarily included in commercial packages of diagnostic products, that contain information about the methods, usage, storage, handling, and/or warnings concerning the use of such diagnostic products. In the same way the term “imprints” is used to refer to instructions and/or information customarily imprinted on commercial packages of diagnostic products that contain information about the methods, usage, storage, handling, the contained materials and/or warnings concerning the use of such diagnostic products.

The kit of the present invention may comprise one or more container(s), optionally with a label. Suitable containers include, for example, bottles, vials, and test tubes. The containers may be formed from a variety of materials such as glass or plastic, and are preferably sterilized. The container holds a composition having an active agent or comprising a buffer which is effective for the detection of the risk haplotype M1 or M2. Further container may hold suitable amplification primers (for example PCR-primers) which allow the specific amplification of the human ANXA5 promoter or the fragments therefore which fragments have been defined herein elsewhere. It is also envisaged that containers are included which hold diverse buffers, for example amplification buffers, and/or buffers for the formation of heteroduplexes etc. The active agent in the composition is preferably an IHG, a positive control (for example an already isolated ANXA5 promoter or a fragment thereof), a negative control, a PCR-amplification enzyme such as the Taq enzyme etc. The kit may further comprise amplification primer pairs for the specific amplification of the human ANXA5 promoter (and in particular for the amplification of at least the fragment of the human ANXA5 promoter). The label on the container indicates that the composition is used for the detection of the risk haplotype M1 or M2 and/or for the amplification of the human ANXA5 promoter, and may also indicate directions for in vitro use, such as those described above.

In a further aspect, the present invention relates to the kit disclosed herein further comprising a nucleic acid sequence comprising a human annexin A5 (ANXA5) promoter or a fragment thereof, which promoter or fragment thereof comprises less than four of the following four nucleotide exchanges

(1) G to A at a position which corresponds to nucleotide 186 of SEQ ID No. 2,
(2) A to C at a position which corresponds to nucleotide 203 of SEQ ID No. 2
(3) T to C at a position which corresponds to nucleotide 229 of SEQ ID No. 2, and
(4) G to A at a position which corresponds to nucleotide 276 of SEQ ID No. 2;

The above embodiment discloses several possible positive controls of the human ANXA5 promoter (including fragments thereof) which comprise less then the four, i.e. three, two or just one or even none of the nucleotide exchanges defined herein.

The present invention also relates to the kits as disclosed herein, wherein said means comprises an IHG. Said IHG is capable of forming specific heteroduplexes with the intended target molecule which “target molecule” is in the context of the present invention the human ANXA5 promoter or a fragment thereof.

In an even further aspect, the present invention relates to the kit defined above, further comprising one or more primer pair(s) capable of amplifying at least a stretch of the human ANXA5 promoter, which stretch comprises at least one of the nucleotide exchanges

(1) G to A at a position which corresponds to nucleotide 186 of SEQ ID No. 2,
(2) A to C at a position which corresponds to nucleotide 203 of SEQ ID No. 2
(3) T to C at a position which corresponds to nucleotide 229 of SEQ ID No. 2, and
(4) G to A at a position which corresponds to nucleotide 276 of SEQ ID No. 2.

It is preferred that the stretches have a length of at least 20 consecutive nucleotides. Preferably said stretches cover nucleotide exchanges (2) and (3) defined herein, more preferably all four nucleotide exchanges (1), (2), (3), and (4), defined above.

The present invention also relates to the use of the nucleic acid sequences or fragments thereof, and/or the IHG(s), and/or the primer pair(s), disclosed herein for the manufacture of a kit or diagnostic composition, for use in a diagnostic method of the present invention.

This disclosure may best be understood in conjunction with the accompanying drawings, incorporated herein by references. Furthermore, a better understanding of the present invention and of its many advantages will be had from the following examples, given by way of illustration and are not intended as limiting.

The figures show:

FIG. 1. (a) Scheme of the N and M2 ANXA5 allelic differences on the DNA and mRNA level and amplification strategy from cDNA. utr Exon1, non-translated Exon 1, tsp, transcription start point; *, nucleotide count start; 1., 2., 3., variant ANXA5 transcripts; forward primer G anneals to all N ANXA5 transcripts, forward primer A anneals to all M2 ANXA5 transcripts. (b) N/M2 allele specific PCR does not generate product from N/N samples, as demonstrated by direct visualization on 1.5% agarose gel. Arrow points at the specific amplicon band of 131 by in the N/M2 control lane, asterisks below the N/N lanes denote non-utilized primers. (c) Melting curve analysis of N/M2 M2 allele-specific products. Melting curves are homogenous at ca. 93° C., indicative of a single amplicon. Base line non-specific products appear only in water samples at ca. 82° C.

FIG. 2 (a) Expression ratio of ANXA5 N allele mRNA in heterozygous (N/M2) vs normal homozygous (N/N) placentas. Output was generated with the REST (2008) software. (b) Expression ratio of ANXA5 M2 allele mRNA to N allele mRNA in heterozygous (N/M2) placentas. Output was generated with the 2^{DDCt} method. (c) Typical western blot of placental protein samples. Blots were cut in strips around the relative molecular mass for α-tubulin (55 kDa) and annexin A5 (36 kDa). Lanes in upper rows were probed with anti-α-tubulin antibodies to ensure equal protein loading and lower row lanes were probed with anti annexin A5 antibodies. Placental genotypes are indicated above each lane.

FIG. 3 Illustration of SEQ ID No. 2

FIG. 4 Structure of the ANXA5 core promoter region. The region boundaries are marked with vertical bars and are numbered according to the position of the first transcription start point (tspl). Untranslated exon 1 is shaded in gray. Transcription factor consensi are in small print and abbreviations of corresponding transcription factors are italisized over the sequence rows. NotI and BamHI restriction sites are underlined and the sequence of the Z-DNA stretch in the promoter is in italics. Nucleotides marking transcription start points (tsp) are underlined. Regions important for promoter function (motifs A and B accordingly) occupy nucleotide positions 295-311 and 328-337. Nucleotides changed in the BamHI; haplotype are bolded and substituting nucleotides are indicated in bold capital letters over the matching respective positions.

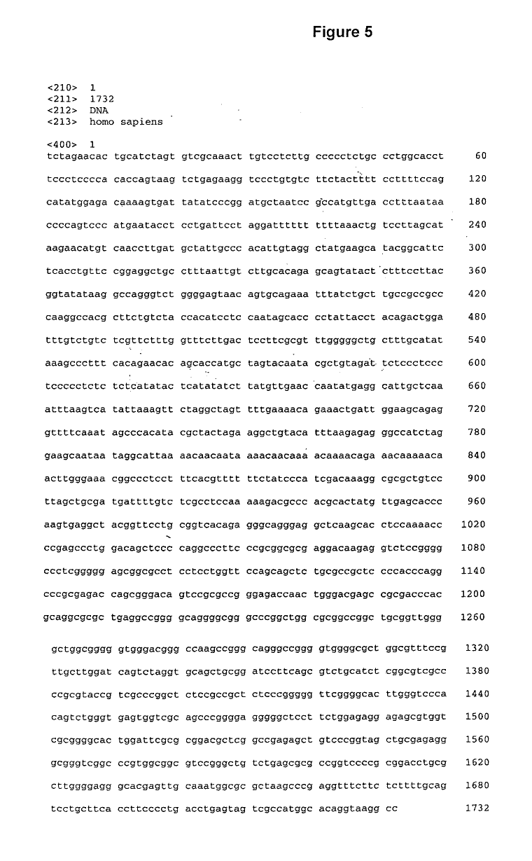

FIG. 5 Illustration of SEQ ID No. 1

FIG. 6 Illustration of SEQ ID No. 3

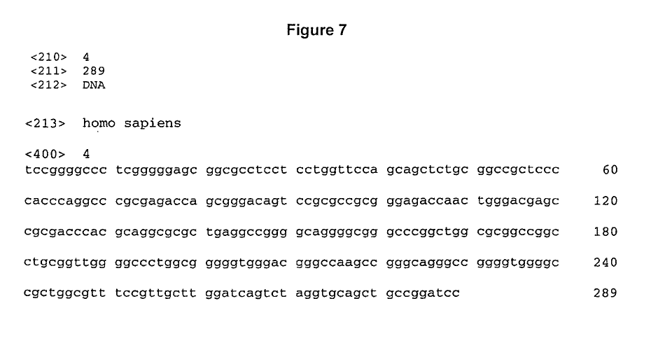

FIG. 7 Illustration of SEQ ID No. 4

### EXAMPLES

The following examples illustrate the invention. These examples should not be construed as to limit the scope of this invention. The examples are included for purposes of illustration and the present invention is limited only by the claims.

### Example 1

### Genotyping

Genomic DNA was extracted from maternal blood and lyophilized placenta, as described [11]. Samples were genotyped for M2/ANXA5 as indicated in [8]. Six samples from N/M2 heterozygous placentas and concordant or discordant maternal genotypes (2 cases and 1 control N/M2, 2 cases M2/M2 and 1 case N/N) and 16 N/N samples (13 cases and 3 controls) were analyzed from 18 PE and/or FGR cases and 4 controls.

### Example 2

### DNA, RNA and protein extraction

DNA, RNA and protein were extracted from 20 mg of the selected samples using the AllPrep DNA/RNA/Protein Mini Kit (Qiagen, Hilden, Germany). Quantitative real time PCR (qRT PCR) Diluted reverse-transcribed cDNA were used for subsequent qRT PCR, with the Light Cycler 480 SYBR Green I Master Mix (Roche Applied Sciences, Mannheim, Germany) and gene specific primers. Amplifications were performed and quantitatively evaluated on a Light Cycler 480 System (Roche Applied Sciences, Mannheim, Germany), employing recommended conditions. For all samples a control housekeeping gene, TBP [12], amplicon was generated [13]. The N allele of ANXA5 was amplified in all samples using primers 5′CAGTCTAGGTGCAGCTGCCG3′ and 5′GGTGAAGCAGGACCAGACTGT3′ and annealing 65° 83 C, and the M2 allele was amplified in N/M2 samples with 5′CAGTCTAGGTGCAGCTGCCA3′ and 5′GGTGAAGCAGGACCAGACTGT3′ and annealing at 63° 85 C. Allelic mRNA quantities were normalized to TBP mRNA and the expression of N alleles in heterozygous samples N/M2 was compared to N/N using the REST software (REST 2008 V2.0.7, Corbett Research, Sydney, Australia). The strong PCR efficiencies correlation of the N and M2 alleles allowed the use of the 2^{DDCt} method [14] for normalized allelic mRNA quantitative comparisons in N/M2 samples. Two independent assays were performed in triplicates for each amplicon.

### Example 3

### Western Blot Analysis and Protein Quantification

Proteins were analyzed using standard Western blot protocols (ECLplus, GE Healthcare, Freiburg, Germany). Annexin A5 was visualized using sheep anti-annexin A5 antibodies [15].

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, detailed Description, and Examples is hereby incorporated herein by reference.

### REFERENCES

[1] Gerke V, Creutz, C E, Moss S E. Annexins: linking Ca2+ signaling to membrane dynamics. Nat Rev Mol Cell Biol 2005; 6: 449-61.
[2] Thiagarajan P, Tait, J F. Binding of annexin V/placental anticoagulant protein I to platelets. Evidence for phosphatidylserine exposure in the procoagulant response of activated platelets. J Biol Chem 1990; 265: 17420-3.
[3] Ravassa S, Bennaghmouch A, Kenis H, Lindhout T, Hackeng T, Narula J, Hofstra L, Reutelingsperger C. Annexin A5 down-regulates surface expression of tissue factor: a novel mechanism of regulating the membrane receptor repertoir. J Biol Chem 2005; 280: 6028-35.
[4] Morgan R O, Bell D W, Testa J R, Fernandez M P. Genomic locations of ANX11 and ANX13 and the evolutionary genetics of human annexins. Genomics 1998; 48: 100
[5] Carcedo M T, Iglesias J M, Bances P, Morgan R O, Fernandez M P. Functional analysis of the human annexin A5 gene promoter: a downstream DNA element and an upstream long terminal repeat regulate transcription. Biochem. J 2001; 356: 571-9.
[6] Rand J H, Wu X X, Guller S, Gil J, Guha A., Scher J, Lockwood C J. Reduction of annexin-V (placental anticoagulant protein-I) on placental villi of women with antiphospholipid antibodies and recurrent spontaneous abortion. Am J Obstet Gynecol 1994; 171: 1566-72.
[7] Shu F, Sugimura M, Kanayama N, Kobayashi H, Kobayashi T, Terao T. Immunohistochemical study of annexin V expression in placentae of preeclampsia. Gynecol Obstet Invest 2000; 49: 17-23.
[8] Bogdanova N, Horst J, Chlystun M, Croucher P J, Nebel A, Bohring A, Todorova A, Schreiber S, Gerke V, Krawczak M, Markoff A. A common haplotype of the annexin A5 (ANXA5) gene promoter is associated with recurrent pregnancy loss. Hum Mol Genet 2007; 16: 573-78.
[9] Chinni E, Tiscia G L, Colaizzo D, Vergura P, Margaglione M, Grandone E. Annexin V expression in human placenta is influenced by the carriership of the common haplotype M2. Fertil Steptil 2009; 91: 940-2.
[10] Tiscia G, Colaizzo D, Chinni E, Pisanelli D, Scianname N, Favuzzi G, Margaglione M, Grandone E. Haplotype M2 in the annexin A5 (ANXA5) gene and the occurrence of 171 obstetric complications. Thromb Haemost 2009; 102: 309-13.
[11] Chinni E, Colaizzo D, Margaglione M, Rubini C, D'Ambrosio R L, Giuliani F, Di Vagno G, Grandone E. Correlation between factors involved in the local haemostasis and angiogenesis in full term human placenta. Thromb Res 2008; 122: 376-82.
[12] Meller M, Vadachkoria S, Luthy D A, Williams M A. Evaluation of housekeeping genes in placental comparative expression studies. Placenta 2005; 26: 601-77
[13] Bièche I, Onody P, Tozlu S, Driouch K, Vidaud M, Lidereau 180 R. Prognostic value of ERBB family mRNA expression in breast carcinomas. Int J Cancer 2003; 106: 758-65.
[14] Livak K J, Schmittgen T D. Analysis of relative gene expression data using real-time quantitative PCR and the 2(-Delta Delta C(T)) Method. Methods 2001; 25: 402-8
[15] Markoff A, Bogdanova N, Knop M, Rüffer C, Kenis H, Lux P, Reutelingsperger C, Todorov V, Dworniczak B, Horst J, Gerke V. Annexin A5 interacts with polycystin-1 and interferes with the polycystin-1 stimulated recruitment of E-cadherin into adherens junctions. J Mol Biol 2007; 369: 954-66.
[16] Sifakis S, Soufla G, Koukoura O, Soulitzis N, Koutroulakis D, Maiz N, Konstantinidou A, Melissari E, Spandidos D A. Decreased annexin A5 mRNA placental expression in pregnancies complicated by fetal growth restriction. Thromb Res 2010; 125: 326-31.

1. A method for diagnosing or detecting a predisposition of a female subject to recurrent pregnancy loss (RPL), preeclampsia (PE) and/or fetal growth restriction (FGR), comprising:
(a) examining the human annexin A5 (ANXA5) promoter in a non-maternal sample, in particular a sample obtained from the (intended) biological father, or in a chorion biopsy or amniocentesis sample obtained from said female, or in a single cell sample obtained before or during the morula stadium of an in vitro fertilized embryo prior to its implantation into said female subject, to detect the following nucleotide exchanges
(i)
(1) G to A at a position which corresponds to nucleotide 186 of SEQ ID No. 2,
(2) A to C at a position which corresponds to nucleotide 203 of SEQ ID No. 2
(3) T to C at a position which corresponds to nucleotide 229 of SEQ ID No. 2, and
(4) G to A at a position which corresponds to nucleotide 276 of SEQ ID No. 2;
or
(ii)
(2) A to C at a position which corresponds to nucleotide 203 of SEQ ID No. 2, and
(3) T to C at a position which corresponds to nucleotide 229 of SEQ ID No. 2,
(b) determining whether the nucleotide exchanges defined in (i) and/or (ii) are present,
wherein the presence of the nucleotide exchanges defined in (i) and/or (ii) indicates a predisposition of said female subject to recurrent pregnancy loss (RPL); and wherein the presence of the nucleotide exchanges defined in (i) indicates a predisposition of said female subject to preeclampsia (PE) and/or fetal growth restriction (FGR).

2. The method of claim 1, further comprising the steps of:
(c) examining the human annexin A5 (ANXA5) promoter in a maternal sample obtained from said female subject to detect the nucleotide exchanges defined in (i) and/or (ii) of claim 1, and
(d) determining whether the nucleotide exchanges defined in (i) and/or (ii) of claim 1 are present.

3. The method of claim 1, wherein said female subject is a subject which has been diagnosed to have a predisposition to recurrent pregnancy loss (RPL), preeclampsia (PE) and/or fetal growth restriction (FGR), by way of examining the human annexin A5 (ANXA5) promoter in a maternal sample obtained from said female to detect the nucleotide exchanges defined in (i) or (ii) of claim 1.

4. The method of claim 1, wherein said female subject is a subject which has been diagnosed not to have a predisposition to recurrent pregnancy loss (RPL), preeclampsia (PE) and/or fetal growth restriction (FGR), by way of examining the human annexin A5 (ANXA5) promoter in a maternal sample obtained from said female to detect the nucleotide exchanges defined in (i) or (ii) of claim 1.

5. The method of claim 1, wherein said sample obtained from the intended biological father, the biological father, and/or the maternal sample is a blood sample, a tissue sample, or a cell sample.

6. The method of claim 1, wherein the detection of the nucleotide exchanges defined in (i) and/or (ii) of the preceding claims, is carried out by nucleic acid detection techniques.

7. The method of claim 6, wherein the detection of the nucleotide exchanges defined in (i) and/or (ii) is carried out by a method for genotyping a target gene sequence associated with an inherited genetic disorder, comprising:
(a) providing a population of induced heteroduplex generator (IHG) molecules corresponding to said target gene sequence, the IHG molecule being a synthetic DNA sequence including at least one nucleotide position which corresponds to a known polymorphic site in the genomic DNA sequence of the target gene sequence and at least one nucleotide substitution, deletion and/or insertion (“identifier”) relative to the genomic sequence at a nucleotide position spaced by a distance of at least one base from the nucleotide position which corresponds to the known polymorphic site and wherein the nucleotide(s) between the nucleotide position which corresponds to the polymorphic site and the identifier are unchanged from the genomic sequence; wherein the at least one identifier comprises an insertion of one or more bases and the IHG molecule is selected to provide improved separation of the resolved bands by a method comprising comparing the separation obtained using the IHG molecule with the separation obtained using a corresponding IHG in which the identifier is not so spaced;
(b) providing a population of the target gene sequence;
(c) combining the respective populations of (a) and (b) under conditions suitable for heteroduplex formation, to obtain induced heteroduplexes between the target gene sequence and an IHG molecule corresponding to said target gene sequence;
(d) resolving the induced heteroduplexes into bands on a suitable support; and
(e) analysing the resolved induced heteroduplexes to determine the genotype of the target gene sequence.

8. A nucleic acid sequence comprising a human annexin A5 (ANXA5) promoter, which promoter comprises the nucleotide exchanges defined in (i) or (ii) of claim 1.

9. The nucleic acid sequence of claim 8, which is under conditions suitable for the amplification of said nucleic acid sequence.

10. The nucleic acid sequence of claim 9, wherein said amplification is carried out by way of PCR.

11. The nucleic acid sequence of claim 8, which is under conditions suitable for heteroduplex formation.

12. Kit for use in a diagnostic method of claim 1, said kit comprising:
(a) a package insert and/or an imprint indicating that said kit is to be employed in a method of claim 1; and
(b) means to carry out the methods of claim 1; and
(c) optionally the nucleic acid sequence of claim 8.

13. The kit of claim 12 further comprising a nucleic acid sequence comprising a human annexin A5 (ANXA5) promoter, which promoter comprises less than four of the following four nucleotide exchanges
(1) G to A at a position which corresponds to nucleotide 186 of SEQ ID No. 2,
(2) A to C at a position which corresponds to nucleotide 203 of SEQ ID No. 2
(3) T to C at a position which corresponds to nucleotide 229 of SEQ ID No. 2, and
(4) G to A at a position which corresponds to nucleotide 276 of SEQ ID No. 2;

14. The kit of claim 12, wherein said means defined in claim 12(b) comprises an IHG.

15. The kit of claim 12, further comprising one or more primer pair(s) capable of amplifying at least a stretch of the nucleic acid sequence defined in claim 8, which stretch comprises at least one of the nucleotide exchanges identified in claim 8.


SEQUENCE LISTING (8 sequences)

SEQ ID NO: 1 - DNA, 1732 bp, Homo sapiens
tctagaacactgcatctagtgtcgcaaacttgtcctcttgccccctctgccctggcaccttccctccccacaccagtaagtctgagaaggtccctgtgtcttctacttttccttttccagcatatggagacaaaagtgattatatcccggatgctaatccgccatgttgacctttaataaccccagtcccatgaatacctcctgattcctaggattttttttttaaactgtccttagcataagaacatgtcaaccttgatgctattgcccacattgtaggctatgaagcatacggcattctcacctgttccggaggctgcctttaattgtcttgcacagagcagtatactctttccttacggtatataaggccagggtctggggagtaacagtgcagaaatttatctgcttgccgccgcccaaggccacgcttctgtctaccacatcctccaatagcacccctattacctacagactggatttgtctgtctcgttctttggtttcttgactccttcgcgtttgggggctgctttgcatataaagccctttcacagaacacagcaccatgctagtacaatacgctgtagattctccctccctccccctctctctcatatactcatatatcttatgttgaaccaatatgaggcattgctcaaatttaagtcatattaaagttctaggctagttttgaaaacagaaactgattggaagcagaggttttcaaatagcccacatacgctactagaaggctgtacatttaagagagggccatctaggaagcaataataggcattaaaacaacaataaaacaacaaaacaaaacagaaacaaaaacaacttgggaaacggccctcctttcacgttttttctatcccatcgacaaaggcgcgctgtccttagctgcgatgattttgtctcgcctccaaaaagacgcccacgcactatgttgagcacccaagtgaggctacggttcctgcggtcacagagggcagggaggctcaagcacctccaaaaccccgagccctggacagctccccaggcccttcccgcggcgcgaggacaagaggtctccggggccctcgggggagcggcgcctcctcctggttccagcagctctgcgccgctccccacccaggcccgcgagaccagcgggacagtccgcgccgggagaccaactgggacgagccgcgacccacgcaggcgcgctgaggccggggcaggggcgggcccggctggcgcggccggctgcggttggggctggcgggggtgggacgggccaagccgggcagggccggggtggggcgctggcgtttccgttgcttggatcagtctaggtgcagctgcggatccttcagcgtctgcatctcggcgtcgccccgcgtaccgtcgcccggctctccgccgctctcccgggggttcggggcacttgggtcccacagtctgggtgagtggtcgcagcccggggagggggctccttctggagaggagagcgtggtcgcggggcactggattcgcgcggacgctcggccgagagctgtcccggtagctgcgagagggcgggtcggcccgtggcggcgtccgggctgtctgagcgcgccggtccccgcggacctgcgcttggggagggcacgagttgcaaatggcgcgctaagcccgaggtttcttctcttttgcagtcctgcttcaccttcccctgacctgagtagtcgccatggcacaggtaaggcc

SEQ ID NO: 2 - DNA, 281 bp, Homo sapiens
tccggggccctcgggggagcggcgcctcctcctggttccagcagctctgcgccgctccccacccaggcccgcgagaccagcgggacagtccgcgccgggagaccaactgggacgagccgcgacccacgcaggcgcgctgaggccggggcaggggcgggcccggctggcgcggccggctgcggttggggctggcgggggtgggacgggccaagccgggcagggccggggtggggcgctggcgtttccgttgcttggatcagtctaggtgcagctgcggatcc

SEQ ID NO: 3 - DNA, 436 bp, Homo sapiens
ccgagccctggacagctccccaggcccttcccgcggcgcgaggacaagaggtctccggggccctcgggggagcggcgcctcctcctggttccagcagctctgcggccgctccccacccaggcccgcgagaccagcgggacagtccgcgccgcgggagaccaactgggacgagccgcgacccacgcaggcgcgctgaggccggggcaggggcgggcccggctggcgcggccggcctgcggttggggccctggcgggggtgggacgggccaagccgggcagggccggggtggggccgctggcgtttccgttgcttggatcagtctaggtgcagctgccggatccttcagcgtctgcatctcggcgtcgccccgcgtaccgtcgcccggctctccgccgctctcccgggggttcggggcacttgggtcccacagtctgg

SEQ ID NO: 4 - DNA, 289 bp, Homo sapiens
tccggggccctcgggggagcggcgcctcctcctggttccagcagctctgcggccgctccccacccaggcccgcgagaccagcgggacagtccgcgccgcgggagaccaactgggacgagccgcgacccacgcaggcgcgctgaggccggggcaggggcgggcccggctggcgcggccggcctgcggttggggccctggcgggggtgggacgggccaagccgggcagggccggggtggggccgctggcgtttccgttgcttggatcagtctaggtgcagctgccggatcc

SEQ ID NO: 5 - DNA, 20 bp, Artificial Sequence
  primer
cagtctaggtgcagctgccg

SEQ ID NO: 6 - DNA, 21 bp, artificial sequence
  primer
ggtgaagcaggaccagactgt

SEQ ID NO: 7 - DNA, 20 bp, artificial sequence
  primer
cagtctaggtgcagctgcca

SEQ ID NO: 8 - DNA, 21 bp, artificial sequence
  primer
ggtgaagcaggaccagactgt